(19) United States
(12) Patent Application Publication
(10) Pub. No.: US 2007/0231311 A1
(43) Pub. Date: Oct. 4, 2007

(54) Method for preserving grain by-products

(76) Inventors: Kurt Dubear Kroening, Greenville, WI (US); David Andrew Stringham, Appleton, WI (US)

Correspondence Address: Michael J. Femal;Femal & Associates, 812 N. Belmont Ave., Arlington Heights, IL, 60004-5606, US

(21) Appl. No.: 11/396,726

(22) Filed: Apr. 3, 2006

(51) Int. Cl. A61K 35/74; C12N 1/20

(52) U.S. Cl. 424/93.45; 435/252.9

(57) ABSTRACT

The present invention relates to an improved method for preserving grain by-products, aka “co-products,” such as wetcake, mash, silage, brewers' cake, and fine distiller's solubles (“syrup”), generated in the production of ethanol products and industrial grade ethanol (“ethanol process”), comprising treating these materials with an effective amount of probiotics, or lactic acid, bacteria, and inhibiting mold growth.

The present invention relates to an improved method for preserving grain by-products, aka “co-products,” such as wetcake, mash, silage, brewers' cake, and fine distiller's solubles (“syrup”), generated in the production of ethanol products and industrial grade ethanol (“ethanol process”), comprising treating these materials with an effective amount of probiotic, or lactic acid, bacteria, and inhibiting mold growth.

### BACKGROUND OF THE INVENTION

Ethanol production from corn, other grains or any plant material containing carbohydrates is accomplished by fermentation using strains of the yeast, Saccharomyces cerevisiae. The ethanol process results in three main products, or co-products: ethanol (used as a fuel additive), carbon dioxide (used as an industrial gas), and a third product, distiller's grains (“DG”), consisting of coarse wet distiller's grains (or wetcake) and a suspension of fine distiller's solubles (or syrup) that contain the leftovers of the fermentation process (mostly water and a smaller portion by weight of reconstituted solids from the original corn kernels). The wet distiller's grains and solubles (“WDGS”), although high in certain nutrients and suitable as feed for cows and cattle, is currently difficult to distribute and use long distances as it gets moldy within 3-4 days in warm weather. High in nutrient value, the wetcake, sometimes with syrup mixed in, is commonly sold and distributed to farmers located near the ethanol production facility for feed, where mold contamination and spoilage associated with lengthy transportation and storage pose less of a problem.

Preventing mold growth on wetcake would enable distribution of the wetcake, the solubles, or WDGS to a far wider geographic area than is currently possible. Further, inhibiting mold growth on wetcake, solubles, or WDGS enables consideration of a broader range of wet distiller's grains uses beyond just cow and cattle feed, such as feed for other agricultural animals and possibly other value-added uses beyond just feed.

There are many commercially available additives, including, among others, an organic acid complex and various chemical solutions such as sodium bisulfite, that slow mold growth, but only for a very limited time (approximately 5-7 days during the warm season versus approximately 3-4 days for normal mold contamination). Current art has very limited effectiveness, unless the volume and concentration of preservative is sufficient. A major disadvantage of this approach is its cost since large quantities of acid must be used to adequately treat the agricultural byproduct material. In addition, the toxic and corrosive nature of concentrated organic acid can require special handling techniques to protect personnel handling the acidic preservative solution. Therefore, development of an alternative, easy-to-handle, relatively inexpensive mold growth inhibitor that works longer and exhibits no adverse taste impact would have a significant positive impact on the use of wet distiller's grains as feed for domesticated cows and cattle. The impact would be even greater if the inhibitor also provides health benefits to the cows, thus increasing their fitness.

Probiotic treatment is using “good” bacteria (harmless or even beneficial to humans or livestock) to out-compete “bad” microorganisms (disease-causing microorganisms including bacteria, yeast, and mold). This can happen through competition for resources (food availability being an important one) as well as production of substances secreted by the probiotic organisms that prevent establishment of harmful microorganisms. Probiotics are currently used by farmers as beneficial additives to cattle feed, but for reasons related to health, survivability, growth rates, and milk production (California Dairy Dispatch Vol. 13, No. 4, Spring 2004; Abe et. al. J. Dairy Sci. 78:2838-2846, 1995; Nocek et. al. J. Dairy Sci. 85:429-433, 2002). If those same or even similar bacteria can also be used to inhibit mold contamination of distiller's grains and solubles, this probiotic addition will actually provide a double benefit for cows, cattle, and possibly other farm animals. Further, effective preservation of moist distiller's grains and solubles (>14% water content by weight) using probiotic bacteria will enable the wider sale and distribution of the distiller's grains as animal feed before it spoils and enable consideration of a wide variety of expanded uses of distiller's grains beyond use as cow and cattle feed.

Microbial spoilage of the WDGS produced from the ethanol manufacturing process can result in the production of off odors, off flavors, and discoloration, which make the WDGS less palatable for cattle and cow feed. A common source of spoilage organisms is found in storage facilities and on associated processing equipment. These spoilage organisms may be either bacteria or fungi. Common bacterial spoilage organisms include those that are members of the genera Clostridium and Listeria. Fungal spoilage is often caused by members of the genera Aspergillus, Penicillium, Candida, Mucor, and Saccharomyces. Additional spoilage concerns are due to the fact that some molds often produce toxins (mycotoxins such as aflatoxins produced by various species of Aspergillus) that are dangerous to animals being fed the distiller's grains. WDGS have a high moisture content (approximately 63-67%), which make them ideal for the growth of fungi (especially mold), which makes it especially important to prevent mold growth on this feed.

Various approaches have been made to limit the proliferation of those microscopic epiphytes (“air plants” that grow on top of the distillers' grains) and storage organisms responsible for spoilage of the wetcake and syrup, two ethanol process co-products known in the industry as “wet distillers' grains” and “condensed distillers solubles”. Since desirable epiphytes are tolerant of low pH, while spoilage organisms such as Clostridium spp. are not, one simple approach has been to spray the distiller's grains and solubles immediately before the time of initial storage with an organic acid as, for example, propionic acid. A major disadvantage of this approach is its limited effectiveness and cost, since large quantities of acid must be used to adequately treat the large-volume agricultural byproduct material.

Another approach to limiting the growth of undesirable organisms during the storage and use of agricultural materials has been to inoculate these materials with those epiphytes thought to be beneficial to the preservation of the agricultural material such that the epiphytes will be able to out-compete and thereby limit the proliferation of the spoilage organisms. Those organisms that were considered most beneficial in the past have been selected primarily on the basis that they were homofermentative lactic acid producers. The rationale for this approach is that the low pH caused by the lactic acid produced by these organisms would inhibit the growth of spoilage organisms. Strains from species of the genera Lactobacillus, Streptococcus, and Pedicoccus are among those considered to be most beneficial in achieving this type of inhibitory effect.

Lactic acid bacteria (LAB) have often been used in the preservation of agricultural and food products utilizing fermentation processes. For example, Hill, U.S. Pat. No. 4,842,871, discloses a method of preserving agricultural products by treating these products with an effective amount of Lactobacillus plantarum (L. plantarum) or mutants of this bacterium. The patentee states that L. plantarum ATCC 53187 is capable of inhibiting common fungal spoilage organisms that are known to be responsible for the deterioration of agricultural products stored for long periods of time for use as feeds. He goes on to suggest the possibility that the inhibition of these fungi and molds is caused by the production of a toxin or antibiotic-like substance that causes a fungistatic or fungicidal effect on these organisms.

Earlier studies cited by Hill in U.S. Pat. No. 4842871 identify any of the LAB, L. plantarum in particular, for their beneficial use in the fermentation of silage material. Further, the earlier studies all note the dependency of the successful use of LAB in beneficiating the fermentation of silage on the presence of carbohydrates, cellulose or starches.

More recent studies have identified various LABS as being good inoculants for improving the aerobic stability of corn silage (Ranjit and Kung, J. Dairy Sci. 83:526-535, 2000) and preserving crimped wheat grains (Adesogan et. al., J. Dairy Sci. 86:1789-1796, 2003). These studies primarily examined the ability of LAB to compete with chemical additives in order to preserve the silage of the feed, which is normally dependent on the availability of carbohydrates (for LAB to convert to short chain fatty acids) and the anaerobic conditions normally associated with ensiling.

Recent research by Weinberg et. al. (J. Dairy Sci. 87:3386-3397, 2004) has examined the previously observed effect of LAB silage inoculants improving animal performance. Their results indicated that LAB pass from silage to rumen fluid and live, providing probiotic effects in dairy cattle. Further research by the same investigators (J. Appl. Microbiol. 98(3): 662-6, 2005) has shown that LAB silage inoculants can also produce antibacterial activity against detrimental microorganisms (Micrococcus luteus and Pseudomonas aeruginosa), which could provide positive probiotic effects on ruminant performance.

Other studies conducted by Filya (J. Dairy Sci. 86:3575-3581, 2003) have demonstrated a positive effect on aerobic stability and ruminal degradability of low dry matter sorghum and corn silages. Many previous studies have indicated that ensiling works well to preserve moist forage crops, although most of the preservation has been attributed to LAB converting carbohydrates to organic acids under anaerobic conditions. The current invention cannot use the same methodology due to the fact that wetcake has very low (<5%) carbohydrate content and that it is exposed to aerobic conditions.

Presque Isle Cultures has publicly available species of probiotic bacteria such as Lactobacillus acidophilus and Lactobacillus plantarum. The species as purchased do not have the ability to inhibit mold growth under room temperature conditions, but can be adapted and/or mutagenized to select for strains that can inhibit mold growth under the desired conditions.

Despite extensive research into the isolation and development of various microorganisms for use as agricultural product inoculants, application of these methods, or their effective adaptation for use in preserving grain byproducts from ethanol manufacturing, has not occurred. Rapid spoilage of grain byproducts from the ethanol process remains an ongoing problem.

### SUMMARY OF THE INVENTION

In the present invention, grain by-products from the ethanol process that are to be preserved for feed and other uses are treated with an effective amount of L. plantarum or L. acidophilus. Unlike most members of these species, L. plantarum and L. acidophilus are capable of inhibiting common fungal spoilage organisms that are known to be responsible for the deterioration of agricultural products stored for long periods of time for use as feeds. It is possible that the inhibition of these fungi and molds is caused by the production of a toxin or antibiotic-like substance, which causes a fungistatic or fungicidal effect on these organisms. It is also possible that the inhibition of these fungi and molds is caused by the reduction in the pH of the substrate to and below a pH level that inhibits the growth of these organisms that spoil the substrate material. The lowering of the pH by LAB is thought to occur primarily by the production of lactic acid from the fermentation of carbohydrates found in the food source. However, due to the fact that WDGS has a very low carbohydrate concentration, lowering of the pH level would have to occur by another mechanism, if it is happening at all. Therefore, the bacteria described below are likely inhibiting mold growth through a different process than that normally associated with LAB in general.

Thus, the present invention provides a method of treating grain byproducts from the ethanol process to enhance their preservation, which comprises administering to the byproducts an effective amount of L. plantarum and L. acidophilus or mutants thereof, and inhibiting fungal growth.

### DETAILED DESCRIPTION OF THE INVENTION

Spoilage of agricultural products due to deterioration caused by the growth of harmful fungi is a major problem in the agricultural community. The same problem persists in the grain-based ethanol industry, which generates large volumes of grain byproducts in conjunction with the ethanol process. Ethanol process is defined as the production of ethanol products, such as beers, ales, lagers, other fermented beverages and spirits, and industrial grade ethanol. In the method according to the invention, grain byproducts from an ethanol process are treated with an effective amount of L. plantarum, L. acidophilus, or mutants thereof, to enhance preservation of the products and diminish the proliferation of spoilage organisms, such as mold, fungi and spoilage bacteria. Methods of treating the wetcake with the probiotic bacteria would include applications such as spraying a liquid culture or lyophilizing the bacteria to be able to add them in a viable powdered form. Additionally, spores of the probiotic bacteria could be generated provided the organisms had the required genes, or were genetically engineered, to form spores.

The method of the invention is especially useful in that the organism of the invention is non-pathogenic and thereby will not cause disease in an animal consuming the preserved distiller's grains. Unlike many types of preservation, which utilize the application of expensive chemical additives having very limited microbe-inhibiting qualities, the method of the invention relies instead upon the enhancement of colonization by desirable organisms through improving their ability to compete with naturally-occurring spoilage organisms. Controlling these harmful organisms is a major consideration, since many of the organisms associated with spoilage of preserved agricultural products are fungi, which are known to produce potent mycotoxins. These mycotoxins are known to cause illness and even death in animals consuming feed contaminated with these fungi and spoilage bacteria.

### EXAMPLES

Two species of probiotic bacteria, Lactobacillus acidophilus and Lactobacillus plantarum were purchased from Presque Isle Cultures and tested for their ability to inhibit mold growth on bacterial (Luria Broth or LB) plates at room temperature (22 degrees Celsius). The mold, obtained from samples obtained at a local ethanol plant, grew very quickly (two days) and there was no significant difference between mold growing on LB plates with or without the probiotic bacteria.

In order to select for probiotic bacteria that would grow more rapidly at room temperature, liquid cultures were struck out on an LB plate at room temperature and growth was examined the next day. Isolated colonies that were larger were selected and used to inoculate tubes of LB liquid broth. Following overnight growth, bacteria were then struck out again on LB plates and allowed to grow overnight. The following day the largest isolated colonies were identified and used to inoculate liquid media again. This process was completed for forty generations. The resulting evolved L. acidophilus and L. plantarum were then inoculated onto an LB plate and mold was added. Growth of mold was observed over a number of weeks and compared to mold growth with no probiotic bacteria. The results from a typical experiment are shown in Table 1.

TABLE 1

| | Mold detection | Mold overgrowth |
|---|---|---|
| Sample | (days) | (days) |
| Control | 2 | 5 |
| L. acidophilus | 7 | 20+ |
| L. plantarum | 8 | 19 |

Similar experiments were conducted on wetcake in plates. Using the evolved probiotic bacteria, mold growth was inhibited but only for short periods of time (5 days as compared to 2 days without the bacteria). Due to difficulties in using wetcake as a bacterial media, methods were developed to both extract the wetcake nutrients to be used in broth and plates as well as using wetcake unmodified in plates. The extraction procedure consisted of adding 60 grams of wetcake to one liter of distilled water and blending for 5 minutes. The mixture was then boiled for 10 minutes and allowed to cool. Cheesecloth was then used to strain the leftover solids from the liquid so that the liquid portion could be used to make a wetcake extract broth as well as wetcake extract plates.

Since the temperature-evolved probiotic bacteria could not grow effectively on wetcake extract broth or plates, dilutions of LB were added to the wetcake extract in order to provide the bacteria with some of the missing nutrients. Bacteria were then selected for growth on diluted LB/wetcake extract broth and plates, as was done for temperature. Over generations, the concentration of LB was decreased to force the bacteria to adapt to growth on wetcake extract. The bacteria were struck out on diluted LB/wetcake extract plates and two days later the largest colonies were used to inoculate diluted LB/wetcake extract broth. After two days of growth in the broth tubes, the bacteria were then struck out again on dilute LB/wetcake extract plates and allowed to grow for two more days. The largest colonies were then selected to inoculate dilute LB/wetcake extract broth, and the procedure was repeated for 20 generations with the dilution of LB down to 1/16 the normal concentration.

In order to increase the speed of adaptation of the LAB species used to develop this research, temperature evolved L. acidophilus and L. plantarum were mutagenized with ethylmethanesulfonate according to established procedures. After recovery, the mutagenized LAB was plated onto diluted LB/wetcake extract plates to identify mutants that grew better on this media. These mutated bacteria were then put into the selection process to inhibit mold growth on wetcake.

The temperature/wetcake evolved L. acidophilus and L. plantarum were then tested for their ability to inhibit mold growth on unmodified wetcake. The results are shown in Table 2.

TABLE 2

| | Mold detection | Mold overgrowth |
|---|---|---|
| Sample | (days) | (days) |
| Control | 2 | 5 |
| L. acidophilus | 5 | 11 |
| L. plantarum | 5 | 12 |

We claim:

1. A method of preserving grain by-products from the ethanol process, which comprises treating said by-products with an effective amount of probiotic, or lactic acid, bacteria, such as Lactobacillus plantarum, Lactobacillus acidophilus, or effective mutants thereof.

2. The method of claim 1, wherein said grain byproducts are selected from the coarse wet distiller's grains, or wetcake, produced in an ethanol process.

3. The method of claim 1, wherein said grain byproducts are selected from the fine distiller's grains (solubles) suspended in an aqueous solution known as condensed distiller's solubles, or syrup, produced in an ethanol process.

4. The method of claim 1, wherein said grain byproducts are selected from a mixture of the wet distiller's grains and the aqueous solution of suspended fine distiller's grains produced in an ethanol process.

5. The method of claim 1, wherein said grain byproducts are selected from the coarse wet brewer's grains, or brewers' cake, produced in an ethanol process.

6. The method of claim 1, wherein said grain byproducts are selected from the fine brewers' grains suspended in an aqueous solution known as thin stillage, produced in an ethanol process.

7. The method of claim 1, wherein said grain byproducts produced in the ethanol process are derived from corn, sorghum, barley, wheat, oats, rye, potato or any plant material containing carbohydrates.

8. The method of claim 1, wherein said treating comprises applying said probiotic, or lactic acid bacteria, such as Lactobacillus plantarum, Lactobacillus acidophilus, or mutants thereof, to said grain byproducts by mixing or spraying.

9. The method of claim 8, wherein said mixing or spraying uses a bacterial composition comprising probiotic, or lactic acid bacteria, such as Lactobacillus plantarum, Lactobacillus acidophilus, or effective mutants thereof.

10. The method of claim 9, wherein said bacterial composition is in a liquid or dried form.

11. The method of claim 9, wherein said composition comprises additional bacterial strains.

12. A method of suppressing the growth of fungi in grain byproducts that comprises treating said byproducts with an effective amount of probiotic, or lactic acid, bacteria, such as Lactobacillus plantarum, Lactobacillus acidophilus, or effective mutants thereof, that possess a fungal inhibiting property.

13. The method of claim 12, wherein said treating comprises applying said probiotic, or lactic acid bacteria, such as Lactobacillus plantarum, Lactobacillus acidophilus, or mutants thereof, to said grain byproducts by mixing or spraying.

14. The method of claim 13, wherein said mixing or spraying uses a bacterial composition comprising probiotic, or lactic acid bacteria, such as Lactobacillus plantarum, Lactobacillus acidophilus, or effective mutants thereof.

15. The method of claim 14, wherein said bacterial composition is in a liquid or dried form. The dried form can also include bacterial spores, a protective form of some species of bacteria that allows them to withstand adverse conditions.

16. The method of claim 14, wherein said composition comprises additional bacterial strains.

17. A biologically pure culture of an organism selected from the group consisting of:
(a) Lactobacillus plantarum or Lactobacillus acidophilus which both possess a fungal and spoilage bacterial (microbial) inhibiting property;
(b) a mutant derivative of Lactobacillus plantarum which possesses a fungal and spoilage bacterial inhibiting property that is equivalent to said fungal and spoilage bacterial inhibiting property of Lactobacillus plantarum; and
(c) a mutant derivative of Lactobacillus acidophilus which possesses a fungal and spoilage bacterial inhibiting property that is equivalent to said fungal and spoilage bacterial inhibiting property of Lactobacillus acidophilus,
wherein said culture is capable of use in enhancing the preservation of the grain byproducts from an ethanol process.

18. The biologically pure culture of an organism of claim 17 wherein the composition of matter further consists of a biologically pure culture and a carrier.

19. The biologically pure culture of an organism of claim 17 wherein the organism is non-pathogenic thereby inhibiting disease in an animal consuming the preserved distiller's grains of the grain byproducts.

20. The biologically pure culture of an organism of claim 17, wherein unlike many other types of preservation of the grain byproducts, which utilize the application of expensive chemical additives having very limited microbe-inhibiting qualities, the pure culture of the organism relies instead upon the enhancement of colonization by desirable organisms through improving their ability to compete with naturally-occurring spoilage organisms, thereby controlling these harmful spoilage organisms which is a major consideration, since many of the spoilage organisms associated with spoilage of preserved agricultural products are fungi, which are known to produce potent and harmful mycotoxins.
